(19) United States
(12) Patent Application Publication
(10) Pub. No.: US 2020/0376212 A1
(43) Pub. Date: Dec. 3, 2020

(54) OIL-INFUSED ELASTOMERIC THERAPEUTIC DEVICE

(71) Applicant: Hone LLC, Washington, UT (US)

(72) Inventors: Michael Dean DeLeeuw, Washington, UT (US); David Hough Crosby, St. George, UT (US)

(73) Assignee: Hone LLC, Washington, UT (US)

(21) Appl. No.: 16/636,635

(22) PCT Filed: Apr. 2, 2019
(86) PCT No.: PCT/US2019/025461

§ 371 Date: Feb. 4, 2020

Related U.S. Application Data
(60) Provisional application No. 62/727,984, filed on Sep. 6, 2018.
(60) Provisional application No. 62/761,614, filed on Apr. 2, 2018.

(51) Int. Cl. A61M 15/08; A61K 9/00; A61K 47/44

(52) U.S. Cl. CPC A61M 15/085; A61M 2210/0618; A61K 47/44; A61K 9/007

(57) ABSTRACT

A clip device is provided, which is configured to removably attach to a body part of a human or animal user. The clip is of an elastomeric, silicone-based material, and the material is configured to absorb an oil to infuse the clip with the oil. The oil is to be applied to a user through the clip when the clip is attached to the body part of the user. In some instances, the clip device is adapted to connect to the nose of the user by way of the user's nostrils.

This application claims benefit to U.S. Provisional Patent Application Ser. No. 62/761,614, filed Apr. 2, 2018 and U.S. Provisional Patent Application Ser. No. 62/727,984, filed Sep. 6, 2018, both of which are incorporated by reference herein in their entirety.

### BACKGROUND

The present disclosure relates in general to the field of aromatherapy delivery, and more specifically, to wearable aromatherapy devices.

Aromatherapy is based on the well-known effect of the positive sensory feedback humans (and even animals) may experience when exposed to certain fragrances and scents. The beneficial effects, whether medicinal, emotional, or otherwise, are evidenced by the large (and growing) consumer markets for fragrances and scent emitting devices. For instance, consumers spend billions of dollars per year purchasing perfumes, colognes, air fresheners and other scented products that emit a desirable and pleasing fragrance. In addition to fragrances providing a merely desirable aroma, recent developments in holistic medicine suggest that aromatherapy may produce beneficial psychological and physiological responses. In light of the foregoing, varying type of mechanisms are used to assist in delivering scents and aromas to consumers conveniently and inexpensively.

### BRIEF DESCRIPTION OF THE DRAWINGS

FIG. 1 illustrates a diagram showing multiple views of an example ring-shaped clip device in accordance with at least some embodiments.

FIGS. 2A-2F illustrate various configurations, which may be incorporated in example ring-shaped clip devices in some embodiments.

FIGS. 3A-3F illustrate examples of knob elements, which may be incorporated in example ring-shaped clip devices in some embodiments.

FIGS. 4A-4F illustrate additional examples of knob elements, which may be incorporated in example ring-shaped clip devices in some embodiments.

FIG. 5 illustrates an image of a ring-shaped clip device being worn on the nose of a human user in accordance with at least some embodiments.

FIG. 6 illustrates an image of a ring-shaped clip device stored in an example package to aid in infusing the clip device with oil in accordance with at least some embodiments.

FIG. 7 illustrates flow diagrams of example processes for preparing oil-infused silicon devices in accordance with at least some embodiments.

FIGS. 8A-8C illustrate example ear plug devices in accordance with at least some embodiments.

FIG. 9 illustrates an image of an example elastomeric piece infused with oil for insertion in a wearable in accordance with at least some embodiments.

FIG. 10 illustrates an example wearable including a pocket to accept an elastomeric piece infused with oil in accordance with at least some embodiments.

FIG. 11 illustrates example wearable elastomeric devices of varying form factors and sizes infused with oils in accordance with at least some embodiments.

Like reference numbers and designations in the various drawings indicate like elements.

### DETAILED DESCRIPTION OF EXAMPLE EMBODIMENTS

In the following description, numerous specific details are set forth regarding the device and methods of the disclosed subject matter and the environment in which such systems and methods may be deployed or used, etc., in order to provide a thorough understanding of the disclosed subject matter. It will be apparent to one skilled in the art, however, that the disclosed subject matter may be practiced without such specific details, and that certain features, which are well known in the art, are not described in detail in order to avoid complication of the disclosed subject matter. In addition, it will be understood that the embodiments provided below are exemplary, and that it is contemplated that there are other systems and methods that are within the scope of the disclosed subject matter.

A device may be provided for the delivery of oil-based particles through inhalation and/or skin absorption. The device may be utilized to provide aromatherapy, to act to conceal or distract from unpleasant or otherwise unwanted odors (e.g., as may be experienced during medical or dental treatments, during airplane flights, crowded public places, various healthcare applications, tobacco odors, etc.). In some implementations, essential oils, such as plant-based or other naturally occurring oils, may provide benefits in the form of energy, use during massage therapy, exercise (e.g., yoga, jogging, and other fitness activities), relaxation, meditation, homeopathy, aromatherapy, as well as relief for individuals struggling with morning sickness, nausea, sleep deprivation, and/or other unwanted odor-based sensations, etc. Additionally, it can also be used to sit within the ear for users who seek relief from ear infections, aches, ringing, etc.

The device may be constructed of an elastomeric material, which is permeable and permits the device to absorb liquids and then later apply the liquid to a user wearing the device. For instance, the liquid may be an at least partially (or fully) volatile oil or other liquid, and the device may be infused with the liquid. The device may be contained in an air-tight container until ready for use. When the device is removed from the container and worn by the user, the liquid may evaporate and the evaporated liquid particles may be inhaled by the user, may come in contact with the skin, taste, or olfactory sensors of the wearer, among other contact. Contact with the liquid may be to affect a medical, antiviral, antibacterial, homeopathic, psychological, or otherwise therapeutic effect on the user.

In some implementations, the elastomeric material making up at least a portion of the device may be silicone-based. In some implementations, the material of the device may be a silicone with durometer equal to or greater than 50 a and equal to or less than 90 a. In other implementations, the silicone used may have a durometer equal to or less than 120 a. Such silicone may be particularly adapted for the absorption (and later secretion or evaporation) of volatile oils. For instance, the silicone may be dipped, submerged, subject to distillation, or otherwise directly exposed to volatile oils, such as essential oils, to cause the silicone of the device to be infused with the oil. The silicone is also flexible but elastic, in that it naturally returns to its originally formed shape. This can allow the device to function as a clip that may allow the device to be reliably (but removably) connected to a body part of a user so as to deliver the oil (or other liquid) infused within the device.

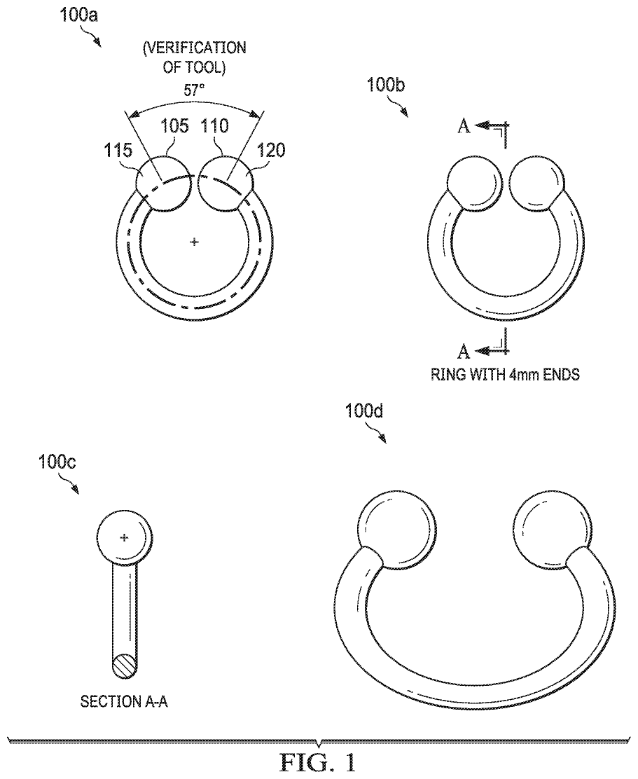

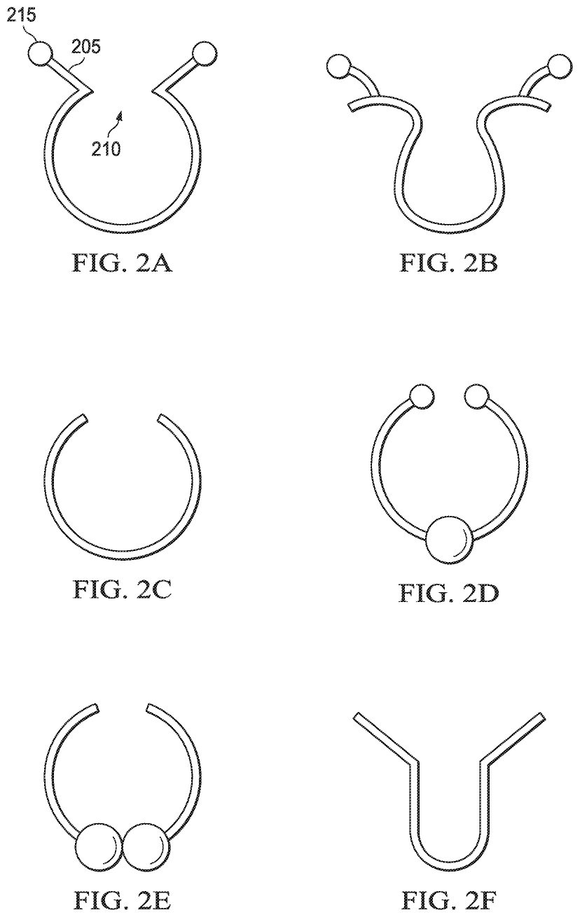

Turning to FIG. 1, a ring-shaped clip device is shown. The clip device may be formed of an elastomeric material, which permits absorption of volatile or partially volatile oils (e.g., such as a silicone of durometer 0 a-120 a). View 100a shows the forward view of the ring-shaped clip. A body part (e.g., the edge of a nostril or the columella of the nose, an ear lobe, lip, etc.) may be positioned between the two (e.g., 105, 110) of the ring clip device and lightly pinched between the two ends, so as to secure the clip device to the body part, among other example configurations (e.g., such as illustrated in FIG. 2A, 2B, 2F). View 100b provide a reference A-A for side view 100c illustrating a cross-sectional view of an example implementation of a ring-shaped clip. View 100b shows that ends (or knobs) of the ring clip device may be 4 mm in diameter. In other implementations, the ends (or knobs) may have different dimensions (e.g., to correspond to the dimensions of a human nostril, animal nostril, etc.). For instance, the diameter of the end or knob may range anywhere between 3-20 mm. In some implementations, such as the example of FIG. 1, such knobs or other protuberances (e.g., 115, 120) may be positioned on the ends of the ring clip so as to further encourage the device to remain in position and provide more volume and surface area for the impregnation of the device with therapeutic oils, which may be applied to the wearer.

Turning to FIGS. 2A-2F, various configurations of ring-like clip devices are shown. For instance, as shown in FIGS. 2A, 2B, and 2F, some ring clips may be enhanced by appendages (e.g., 205) flaring out from the opening (e.g., 210) of the clip. For instance, as shown in FIG. 2A, at the opening 210 of the ring clip, rather than terminating at ends with knobs, the clip can include appendages 205, 210 flaring outwards from the opening. The ends of these appendages, in some implementations, may be provided with knobs (e.g., 215), as in the example of FIG. 2A. The example of FIG. 2B includes multiple appendages which flare out from an opening in the ring clip. More or fewer appendages may be provided in other implementations. Such appendages (e.g., 205) may provide additional support, such as in instances where the ring clip is to be worn in the nose or ear of the user, such that the appendage provides a spring-like pressure on the inside of the nostril or ear canal to encourage the ring clip device to remain in place to more effectively deliver a therapeutic liquid. Further, the bend or corners (e.g., 220) of the opening (e.g., 210) may function as the ends of the ring clip shown in FIG. 1, such that a portion of the body can be pinched between these corners (e.g., 220) to further connect the clip to the portion of the body of the user.

While the examples of FIGS. 2A-2B show a ring clip that includes flared appendages at the opening of the clip and knobs (e.g., 215) provided at the end or tip of one or more of these appendages, in some implementations, knobs may be omitted entirely. For instance, FIG. 2C shows a ring-shaped clip without appendages and FIG. 2F shows a ring clip with flared appendages, both of these designs (in FIGS. 2C and 2F) omitting knobs from the ends of the clip. Further, in some implementations, one or more knobs may be added to the clip at positioned other than the ends of the ring, such as shown in the examples of FIGS. 2D and 2E.

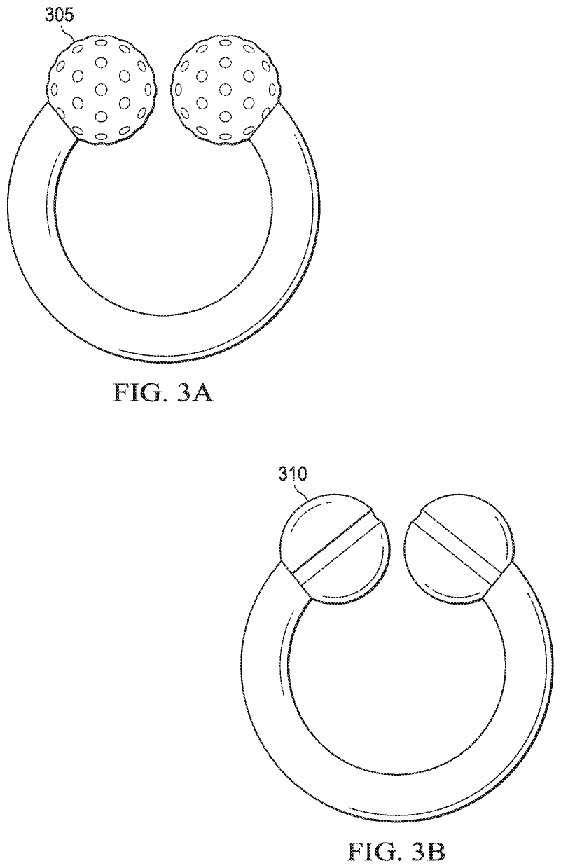

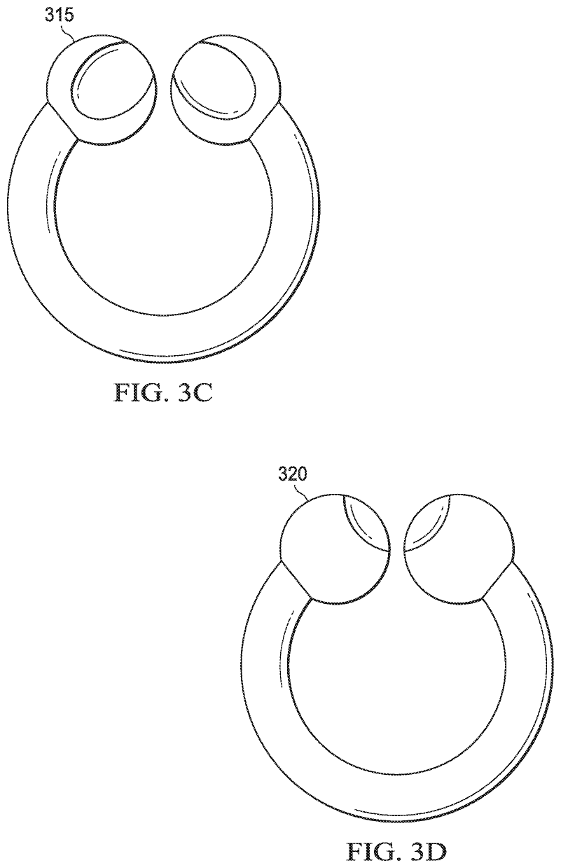

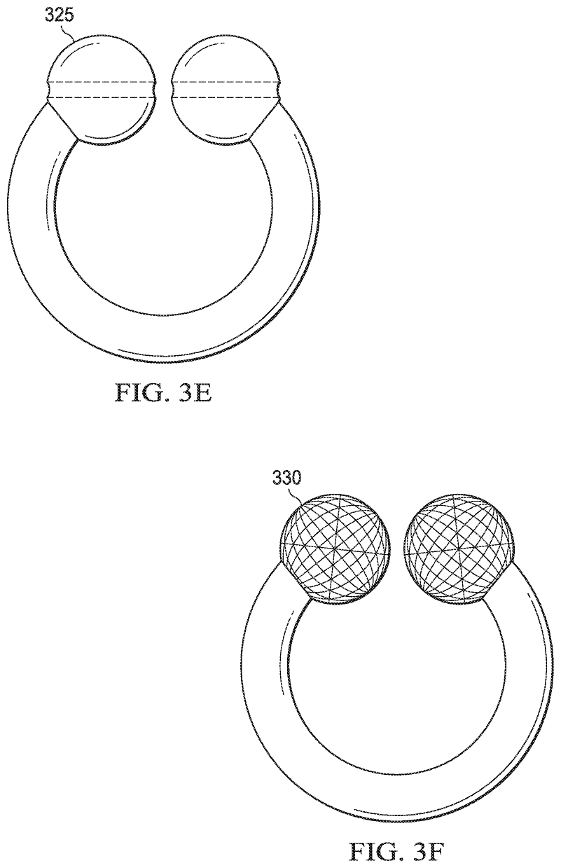

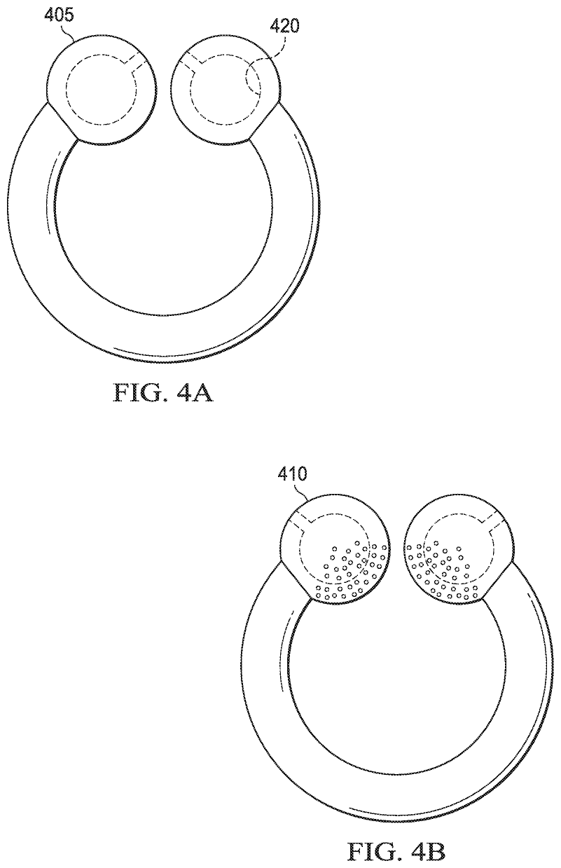

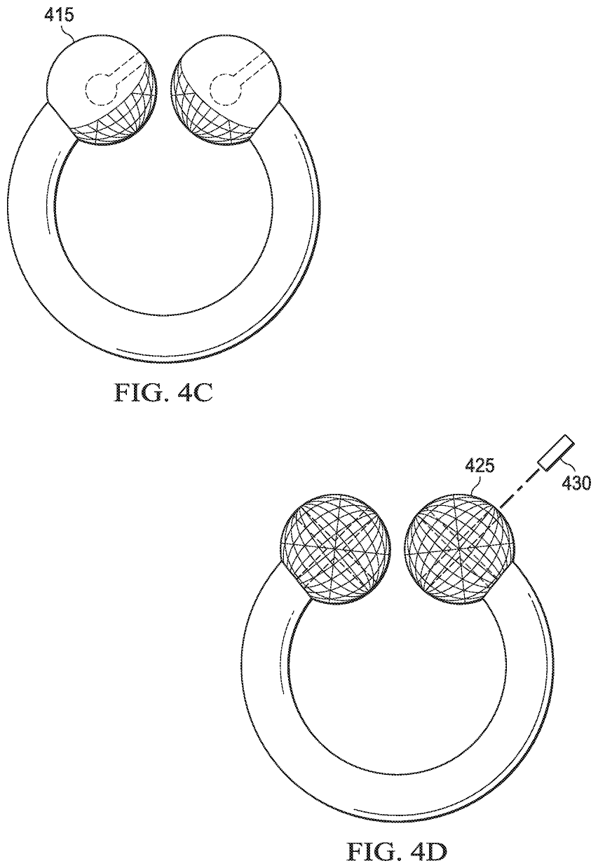

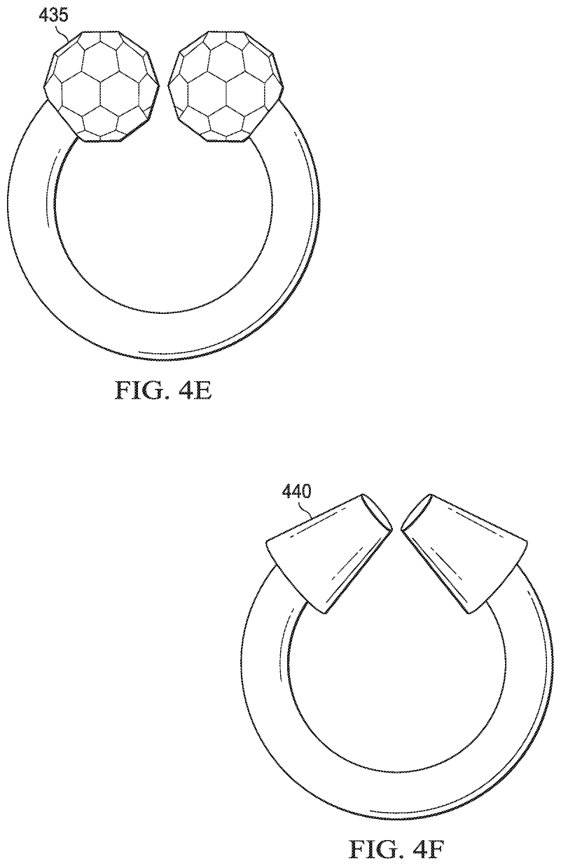

Turning to FIGS. 3A-3F and FIGS. 4A-4F, various implementations are shown illustrating ring clips with various examples of knobs which may be provided on a ring clip design (e.g., any one of the examples of FIGS. 2A-2F). For instance, knobs may be provided which are configured with features to enhance either or both the device's ability to absorb and carry oil and the device's effectiveness in delivering or exposing the oil to the user. For instance, in FIG. 3A, an example knob (e.g., 305) is shown that is provided with dimples over all or a portion of the outside surface of the knob to enhance the amount of oil that may be absorbed and/or carried on the knobs. In the example of FIG. 310, a channel (either formed as a groove on the outside surface of the knob or as a bore or internal channel within the knob) may be provided to carry additional oil (e.g., above and beyond what can be absorbed by the silicon structure of the ring or to enhance the absorption of the oil within the knob (e.g., by injecting oil directly within the channel)). FIG. 3E, shows another example of a channel (implemented as a passthrough bore through the knob (e.g., 325). FIGS. 3C-3D show examples of larger surface depressions or cavities, which may be implemented on example knobs (e.g., 320, 325) to enhance the carrying and delivery of volatile oils impregnated within a ring clip device. Further, as shown in FIG. 3F, rather than providing a solid knob, the knob(s) (e.g., 330) implemented on a ring clip may be formed as a mesh, hollow cavity, starburst, or other geometry to allow excess oil to collect within spaces formed by the non-solid or non-spherical geometry of the knob. Such openings or depressions may also enhance the ability of the oil to evaporate from the knob to be absorbed, ingested, or sensed by the wearer.

Turning to FIGS. 4A-4F, additional examples are shown of implementations of knobs which may be implemented on a ring-shaped clip device. For instance, in FIGS. 4A-4C, cross-sectional views of example knobs 405, 410, 415 are shown that include a cavity (e.g., 420) in which additional oil may be injected for delivery to a user (e.g., above and beyond what is absorbed within the body of the ring clip device). For instance, a syringe may be used to deposit oil within the cavity or reservoir within the knob. In the example of FIG. 4B, small holes may penetrate the reservoir and extend through to the exterior surface of the knob 410 and allow slow release of the oil from the reservoir to the outside of the knob. Similarly, in the example of FIG. 4C, at least a portion of the knob may include a silicone waffle or mesh structure the enwraps a portion of the reservoir to allow oil within the reservoir to leak out for absorption or enhanced evaporation, among other examples.

Turning to FIG. 4D, in some implementations, a knob (e.g., 425) may be enhanced with receptacle to allow a scented element 430 (e.g., an element impregnated with additional oil, such as a felt strip, a different non-silicone insert, another silicone element, or other element) to be removably inserted within the knob. Such inserts may allow users to enhance or modify the experience of the nose clip, such as to enhance or modify the scent provided by the clip (e.g., to create scent mixes or other modifications). Refills of the element 430 may be inserted to replace elements that are expired or to change the scent delivered through the knob, among other examples. In some implementations, an element may include a type of oil different from the oil infused within the body of the ring clip and its corresponding knob (e.g., 425) to allow a combination of oils to be delivered for enhanced therapeutic effect, among other example benefits and uses.

While many of the foregoing examples have illustrated knobs of substantially spherical geometries, it should be appreciated that knobs may be of potentially any suitable geometry or selected among geometries that enhance the ability of the ring clip to attach to a particular body part or to enhance the absorption and/or delivery of therapeutic oils. For instance, as shown in the example of FIG. 4E, a non-spherical geometry may be employed for knobs (e.g., 435) of a clip ring. As another example, shown in FIG. 4F, a substantially conical (or truncated conical) geometry may be adopted for knobs (e.g., 440) of a clip ring, among other example geometries.

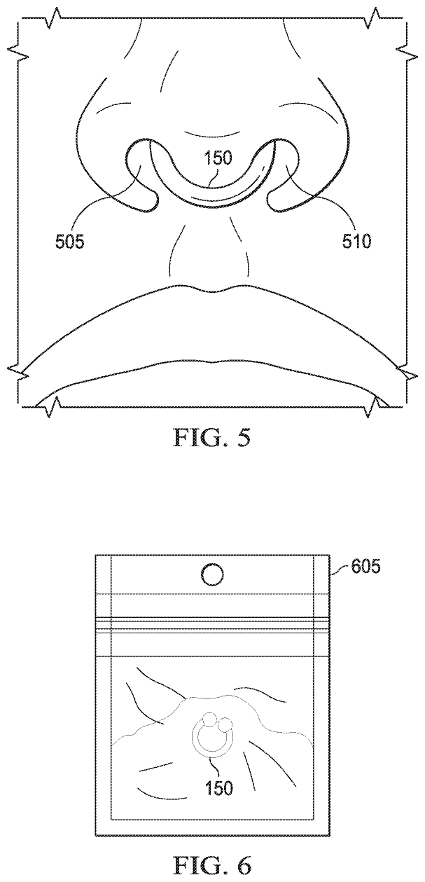

Turning to FIG. 5, a photograph is shown of a ring-like clip device 150 (such as introduced in the foregoing description and figures), being worn by a user. For instance, the clip device 150 may be adapted to be worn by inserting the ends of the clip device within the nostrils 505, 510 of the user, with the elastomeric character of the ring clip lightly pinching the columella of the user. The ring is impregnated with essential oils, which may be volatile at room temperature, causing the scented oil particles to be received by the olfactory sensors of the user. In this manner, therapeutic scents and corresponding oil particle may be administered to the user, either for homeopathic and other therapeutic purposes and/or to mask (i.e., overpower) unwanted odors in the user's environment, among other example uses and benefits.

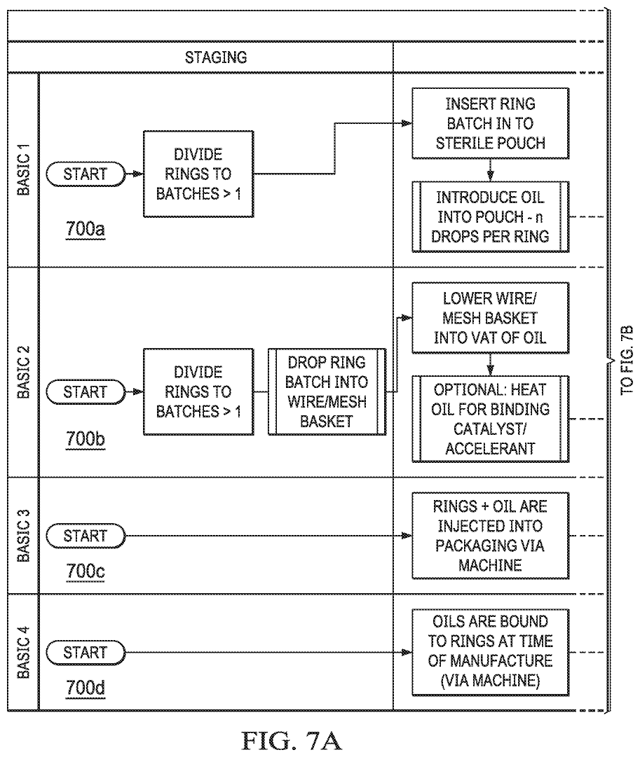

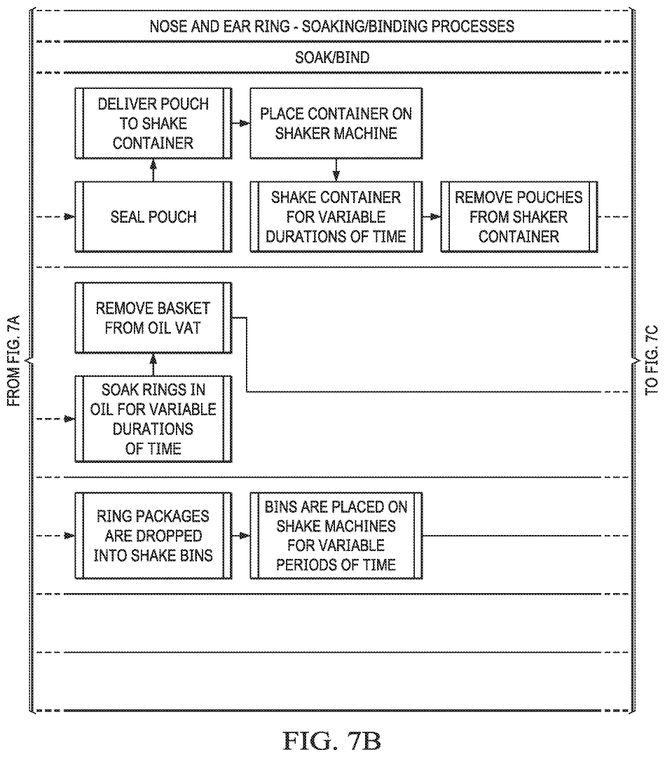

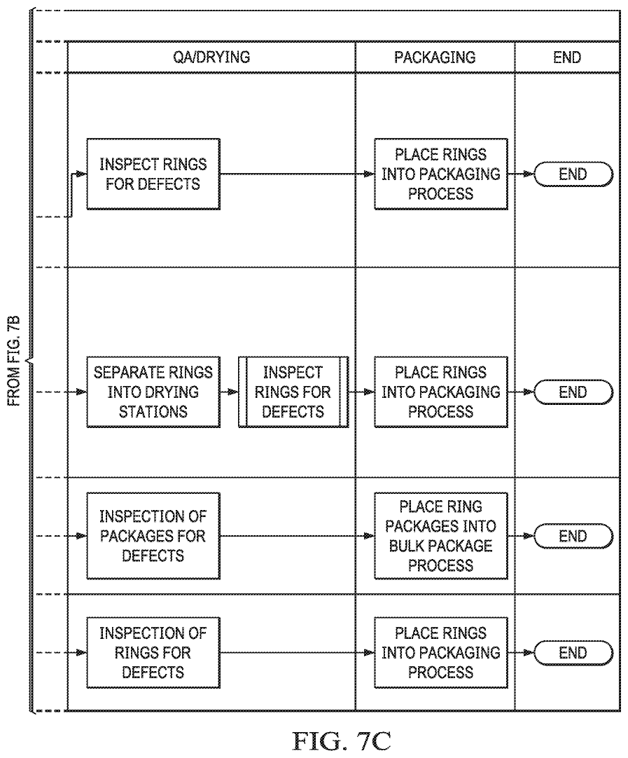

Turning to FIG. 6, an example ring clip device 150 is shown within a mylar package 605. In some implementations, the mylar package may serve as the container for the oil-infused clip device prior to the device's use by a user. The mylar package may also be used to infuse the clip device with oil in some implementations. For instance, a defined amount of oil may be deposited within the package in addition to the clip device. The mylar package may be sealed and the oil may be slowly absorbed into the body of the clip device while awaiting use by a user. Further, when the oil has been consumed through use by a user (e.g., the oil has mostly or fully evaporated from the ring clip), a user may reinfuse the ring clip with oil by depositing additional oil into the mylar package and replacing the clip in the package with the oil for storage (and absorption of the new oil) while the user prepares for the clip's reuse. FIG. 7 illustrates flow diagrams 700a-d illustrating example processes and methods for preparing oil-infused silicon devices using air-tight packages that do not allow the oil to permeate the package material, such as mylar packages.

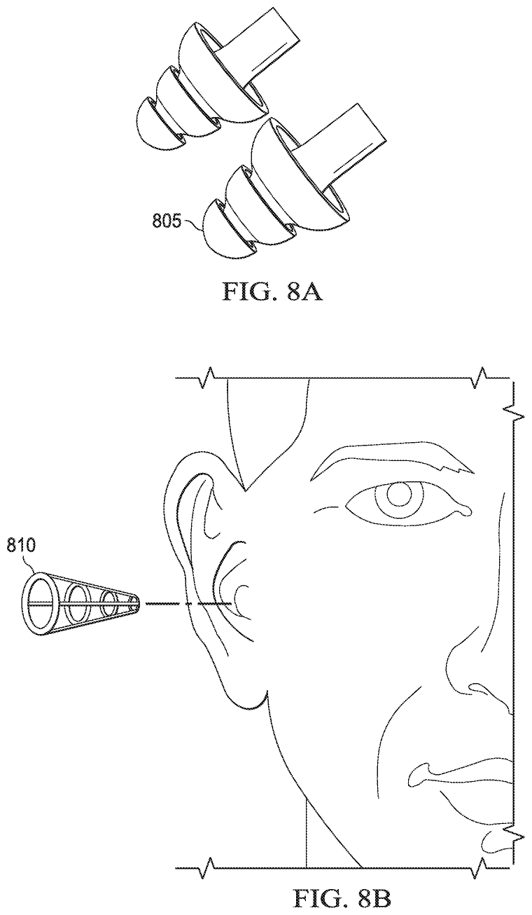

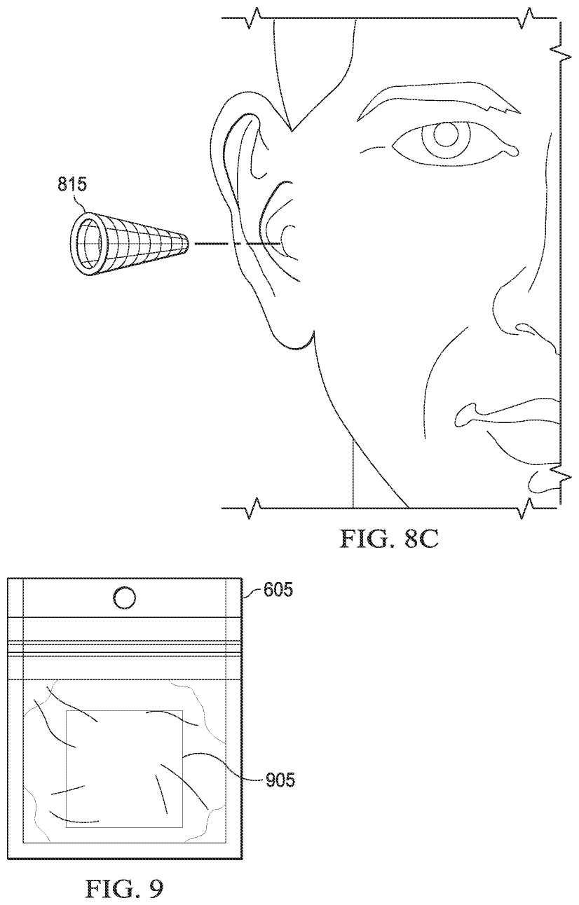

Silicone material possessing characteristics to absorb oil-based liquids (e.g., silicone 0 a-120 a) may be used in other therapeutic devices and form factors than ring-based clip devices. For instance, as shown in the examples of FIGS. 8A-8C, ear plug devices formed from silicone 0 a-120 a may be infused with essential oils to allow deliver the oil particles to the ear canal of a user. For instance, a solid plug-like silicon element 805, such as shown in FIG. 8A may be provided. Other designs (such as shown in FIGS. 8B-8C) may be provided which may allow an oil-infused element (e.g., 810, 815) to be inserted at least partially into the ear cavity or ear canal, while not substantially restricting sound from passing into the user's ear. For instance, a basket-like element 810 may be provided with openings to allow for the passage of sound and which is infused with essential oils for delivery to the user through the user's ear. In another example, shown in FIG. 8C, a hollow element 815 constructed of a silicone mesh maybe infused with essential oil and also allow for sound to pass substantially unrestricted to the user's ear, among other example implementations.

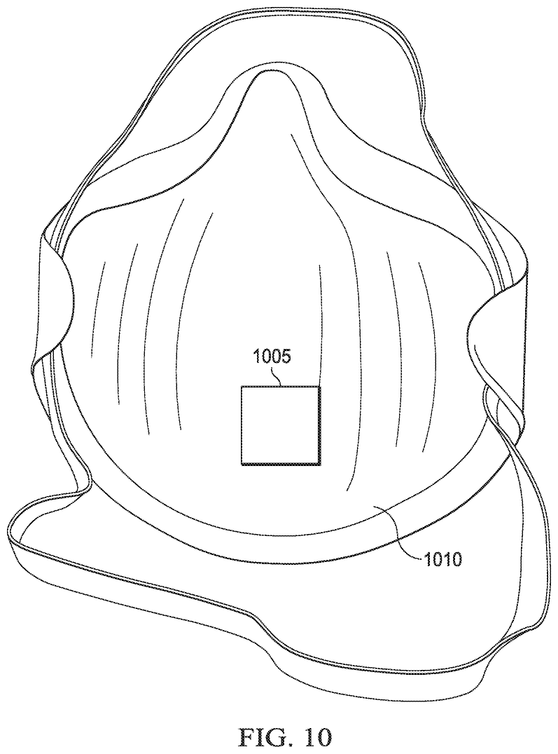

FIG. 9 shows a silicone disk, square, or other piece 905 (e.g., silicone 0 a-120 a) which is infused with essential oils to serve as an element for insertion into a wearable. FIG. 9 is shown with the piece 905 enclosed within a package (e.g., 605), which may aid in infusion of oils into the piece 905. The aromas and evaporated oil particles may be release from this disk through the wearable for administration of the essential oil to the user. For instance, the disk may be attached to the inside of a helmet or mask. For instance, an adhesive backing may be provided to allow oil-infused silicone disks to be removable attached and replaced within/on the wearable. In other implementations, a pouch may be provided in the wearable to allow the insertion and replacement of oil-infused silicone disks. Such pouches may be constructed of mesh or otherwise provide opening so as not to unduly restrict the evaporation and consumption of the oil vapor. FIG. 10 shows an example surgical mask, where an example oil-infused silicone disk 1005 is attached (e.g., through an adhesive, pocket, or other attachment mechanism) to the interior 1010 of the mask, among other example implementations.

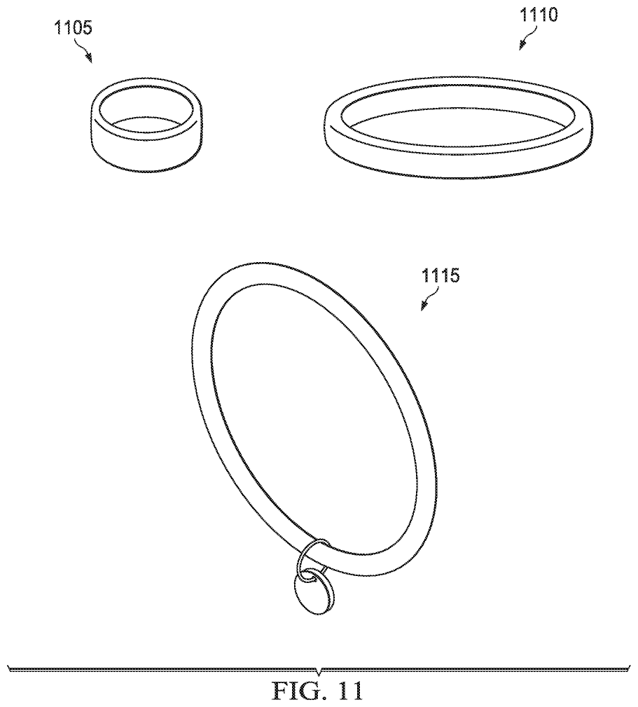

FIG. 11 shows additional examples of silicone-based wearables that may be infused with essential oils for delivery to its wearer. For instance, a silicone ring 1105 may be provided to be worn on the finger of a user. Similarly, oil-infused silicone bracelets (e.g., 1110) and necklaces (e.g., 1115) may be provided to be worn by a user and deliver therapeutic oils to a user. In some implementations, only a portion of the wearable (e.g., a charm on a bracelet or necklace, the knobs of a ring-shaped nose clip, etc.) may be composed of silicone that is infused with volatile oils. In still other examples, other absorbing material may be used to carry essential oils for delivery, through a wearable, to a user. As an example, knobs of a ring clip device may be composed of felt or other absorbent material. In some examples, a mixture of materials (e.g., silicone 0 a-120 a and felt) may be employed in the wearable and infused with therapeutic oils, among other examples.

In still other implementations, the elastomeric material utilized to construct the example wearable devices discusses herein, may be additionally utilized in other implementations to provide alternative products for delivering essential oils to users. For instance, a silicon device may be constructed with a size and shape adapted to hang the silicon device from a coat hanger, bar, towel rod, faucet, shower head, toilet, urinal, or other element, allowing the silicon device to be hung, after being infused with one or more oils, to provide aromatherapy to persons or animals within a room or environment, in which the silicon device is hung, among other example implementations.

As discussed above, a number of example implementations and features have been described. While some of these examples may appear to describe these features implemented in isolation, it should be appreciated that implementations may combine multiple features described herein within a single device without departing from the scope of this disclosure.

As discussed herein, devices may be infused with volatile or partially volatile essential oils. Below are examples of essential oils and therapies associated with various essential oils. Accordingly, oil-infused wearable devices discussed herein may be utilized to provide medical, homeopathic, and therapeutic treatments of users/patients consistent with the following:
anti-bacterial: thieves, colloidal silver, cinnamon bark, clove, lemon, orange, peppermint, rosemary
weight loss: green apple, grapefruit, cinnamon, ginger, peppermint, lemon, bergamot, sandalwood, lavender, fennel, orange, jasmine.
sinus infections: peppermint, ravintsara, eucalyptus blue, thyme, rosemary, blue cypress, dorado azul, eucalyptus radiata, thyme, fennel,
migraines: basil, copaiba, eucalyptus globulus, mastrante, German chamomile, helichrysum, lavender, marjoram, peppermint, rosemary, wintergreen.
headaches: peppermint, clove, copaiba, eucalyptus, globulus, eucalyptus blue, dorado azul, mastrante, German chamomile, lavender, myrrh, roman chamomile rosemary, spearmint, valerian, wintergreen.
tension headache: valerian, cardamom, tangerine, jasmine, palmarosa, mastrante, geranium, sacred frankincense, frankincense, peppermint, lavender, roman chamomile
allergy relief: fennel, eucalyptus blue, lavender, ocotea, roman chamomile, peppermint, German chamomile, marjoram, sacred frankincense, frankincense
morning sickness: peppermint, ginger, spearmint, lavender, lemon
nausea: peppermint, ginger, nutmeg, ocotea
stress: lavender, peppermint, roman chamomile,
anxiety: bergamot, lavender,
sleep insomnia: lavender, goldenrod, valerian, roman chamomile, orange, mandarin
energy/focus/motivation: Roman chamomile, spruce, ylang ylang, lavender, mix for motivation. lavender is known to help with mental concentration and mental acuity
cannabis/medical marijuana: cannabidiol (CBD) oils
sexual disfunctions: ylang ylang
ear infections: headaches eucalyptus oils, peppermint,
smoking cessation: cinnamon, clove, nutmeg, peppermint, roman chamomile, clary sage

Reference throughout this specification to “one implementation,” “an implementation,” “an example,” “one example, “an instance”, or “one instance” means that a particular feature, structure, or characteristic described in connection with the implementation is included in at least one implementation of the present invention. Thus, the appearances of the phrases “in one implementation” or “in an implementation” in various places throughout this specification are not necessarily all referring to the same implementation. Furthermore, the particular features, structures, or characteristics may be combined in any suitable manner in one or more implementations.

The following examples pertain to embodiments in accordance with this Specification. Example 1 is a device including: a clip to removably attach to a body part, where the clip is of an elastomeric, silicone-based material, and the material absorbs an oil to infuse the clip with the oil, where the oil is to be applied to a user through the clip when the clip is attached to the body part of the user.

Example 2 may include the features of example 1, where the clip is infused with the oil and oil includes an at least partially volatile oil, where applying the oil to the user includes evaporation of the oil to expose the user to the evaporated oil.

Example 3 may include the features of example 2, where the body part includes the nose of the user, the clip includes two ends, and each of the two ends is to be at least partially inserted into a respective nostril of the nose of the user to attach the clip to the nose and deliver the evaporated oil to olfactory receptors in the nose of the user.

Example 4 may include the features of any one of examples 2-3, where the oil includes an essential oil.

Example 5 may include the features of any one of examples 1-4, where the body part includes the mouth of the user.

Example 6 may include the features of any one of examples 1-5, where the body part includes the ear of the user.

Example 7 may include the features of any one of examples 1-6, where the clip includes two ends and each of the two ends includes a respective knob.

Example 8 may include the features of example 7, where the knobs are substantially spherical in geometry.

Example 9 may include the features of any one of examples 1-8, where the material includes silicone.

Example 10 may include the features of example 9, where the silicone has a durometer less than 120 a.

Example 11 may include the features of any one of examples 9-10, where the silicone has a durometer equal to or less than 90 a.

Example 12 may include the features of any one of examples 9-11, where the silicone has a durometer within a range including 50 a to 90 a.

Example 13 is a method including: depositing a ring-shaped clip device in an airtight container, where the clip device is to removably attach to a body part, the clip is of silicone with a durometer less than or equal to 120 a; depositing oil within the container; and infusing the clip device with the oil while the clip device and oil are within the container.

Example 14 may include the features of example 13, further including shaking the container with the oil and clip device using a machine.

Example 15 may include the features of any one of examples 13-14, where the oil is to be applied to a user through the clip when the clip is attached to the body part of the user.

Example 16 may include the features of any one of examples 13-15, where the container includes a resealable pouch made of a nonabsorbent material.

Example 17 may include the features of example 16, where the nonabsorbent material includes mylar.

Example 18 may include the features of any one of examples 13-17, where the clip device includes the device of any one of examples 1-12.

Example 19 is a method including: connecting a ring-shaped clip device to a body cavity of a user, where the clip device includes two ends, connecting the clip device includes inserting at least one of the two ends into the body cavity, the clip device is infused with an at least semi-volatile oil; and applying the oil to the user at or near the body cavity while the clip device is connected to the user.

Example 20 may include the features of example 19, where the oil includes an essential oil, and the method includes providing a homeopathic treatment to the user through administration of the essential oil using the clip device.

Example 21 may include the features of any one of examples 19-20, where the body cavity includes a nostril of the user.

Example 22 may include the features of any one of examples 19-20, where the body cavity includes an ear canal of the user.

Example 23 may include the features of any one of examples 19-22, where the clip device includes the device of any one of examples 1-12.

Example 24 is a device including: a disk to be attached to a wearable, where the wearable is at least partially cover the face of a user, the disk is of a silicone-based material, and the material absorbs an oil to infuse the disk with the oil, where the oil is to be applied to a user through the disk when the disk is attached to the wearable.

Example 25 may include the features of example 24, where the wearable includes a mask.

Example 26 may include the features of example 25, where the mask includes a surgical mask.

Example 27 may include the features of any one of examples 24-26, where the disk is to be attached using an adhesive.

Example 28 may include the features of example 27, where the adhesive is deposited on the wearable.

Example 29 may include the features of example 27, where the adhesive is deposited on a side of the disk.

Example 30 may include the features of any one of examples 24-26, where the disk is to be inserted into a pocket provided on the wearable.

Example 31 may include the features of any one of examples 24-30, where the oil includes an essential oil.

Example 32 may include the features of any one of examples 24-31, where the material includes silicone with a durometer less than or equal to 90 a.

Example 33 may include the features of any one of examples 24-32, further including the wearable.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

A detailed description has been given with reference to specific exemplary embodiments. It will, however, be evident that various modifications and changes may be made thereto without departing from the broader spirit and scope of the invention as set forth in the appended claims. The specification and drawings are, accordingly, to be regarded in an illustrative sense rather than a restrictive sense. Furthermore, the foregoing use of embodiment and other exemplarily language does not necessarily refer to the same embodiment or the same example, but may refer to different and distinct embodiments, as well as potentially the same embodiment.

1.-33. (canceled)

34. A device comprising:
a clip to removably attach to a nose of a human user, wherein:
the clip is of an elastomeric, silicone-based material with a durometer less than 120 a, and
the material is infused, through absorption, with an oil,
wherein the oil comprises a volatile oil and evaporates at a temperature less than 95 degrees Fahrenheit, and the clip is to apply vapors of the volatile oil through the nose of the user when the clip is attached to the nose of the user.

35. The device of claim 34, wherein the oil evaporates at a temperature less than 75 degrees Fahrenheit.

36. The device of claim 34, wherein the oil comprises an essential oil.

37. The device of claim 34, wherein the clip is to provide aromatherapy to the user.

38. The device of claim 34, wherein the clip comprises two ends, and each of the two ends is to be at least partially inserted into a respective nostril of the nose of the user to secure the clip to the nose and deliver the vapors to olfactory receptors in the nose of the user.

39. The device of claim 38, wherein each of the two ends contacts a respective ala of the nose to secure the clip in the nose of the user.

40. The device of claim 38, wherein each of the two ends contacts the septum of the nose to secure the clip in the nose of the user.

41. The device of claim 38, wherein each of the two ends comprises a respective knob, the clip further comprises a partial ring segment connecting the knobs, and each of the knobs has a cross-sectional dimension wider than a cross-sectional diameter of the partial ring segment.

42. The device of claim 41, wherein the oil is infused in material of each of the respective knobs and the partial ring segment.

43. The device of claim 41, wherein the clip has an open ring shape comprising an opening formed by a distance between the two knobs when the clip is at rest.

44. The device of claim 41, wherein each of the knobs is substantially spherical in geometry.

45. The device of claim 34, wherein the material has a durometer equal to or less than 90 a.

46. The device of claim 45, wherein the material has a durometer within a range comprising 50 a to 90 a.

47. The device of claim 34, further comprising a sealable, airtight container to store the clip after absorption of the oil in the material of the clip.

48. The device of claim 47, wherein the container comprises a resealable mylar pouch.

49. The device of claim 48, wherein the container contains additional oil.

50. A method comprising:
depositing a ring-shaped clip device in an airtight container, wherein the clip device is to removably attach to a human nose, the clip is of silicone with a durometer less than or equal to 120 a;
depositing oil within the container, wherein the oil comprises a volatile oil and evaporates at a temperature less than 95 degrees Fahrenheit; and
temporarily sealing the container with the clip device and oil, wherein the clip device absorbs at least a portion of the oil while the clip device and oil are within the container.

51. The method of claim 50, further comprising shaking the container while the container encloses the oil and clip device using a machine to encourage absorption of the oil by the clip device.

52. The method of claim 50, wherein the container comprises a resealable pouch made of a nonabsorbent material.

53. The method of claim 50, wherein the clip device comprises two ends, each of the two ends is to be at least partially inserted into a respective nostril of a nose of a user to secure the clip to the nose, each of the two ends comprises a respective knob, the clip further comprises a partial ring segment connecting the knobs, each of the knobs is substantially spherical in geometry, and the clip device has an open ring shape comprising an opening formed by a distance between the two knobs when the clip is at rest.
